(19) United States
(12) Plant Patent Application Publication
(10) Pub. No.: US 2003/0093846 P1
(43) Pub. Date: May 15, 2003

(54) Begonia plant named 'Nadine'

(75) Inventor: Jan Man, Amstelveen (NL)

Correspondence Address: Russell D. Orkin, 700 Koppers Building, 436 Seventh Avenue, Pittsburgh, PA, 15219-1818, US

(73) Assignee: Begonia Breeders Association B.V.

(21) Appl. No.: 10/008,292

(22) Filed: Nov. 13, 2001

(51) Int. Cl. A01H 5/00

(52) U.S. Cl. PLT/346

(57) ABSTRACT

A new and distinct begonia plant with yellow-orange colored double flowers.

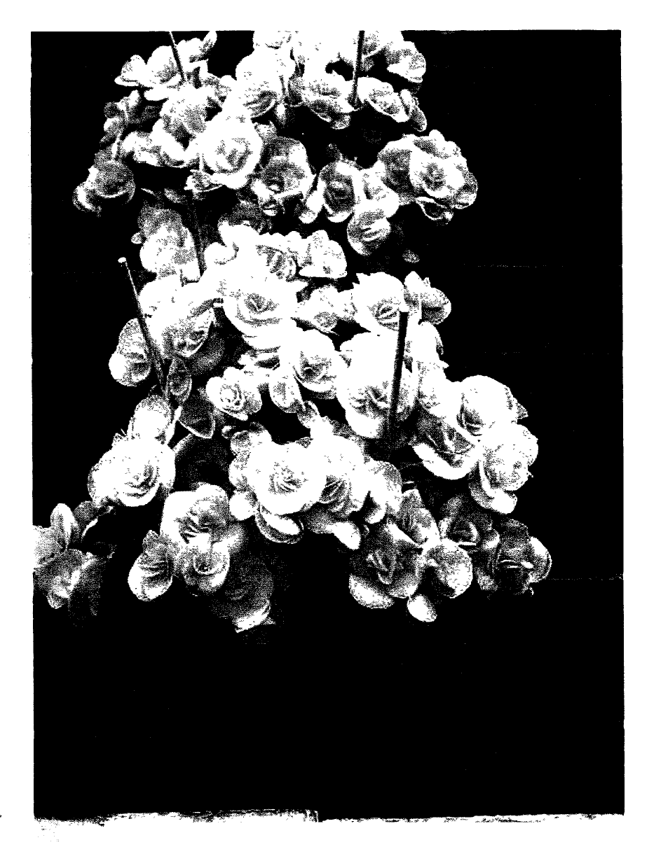

### BACKGROUND OF THE INVENTION

The present invention comprises a new and distinct cultivar of Begonia Elatior known by the varietal name ‘Nadine’. The new variety was discovered in a planned breeding program as a single plant in a group of plants of the parent cultivar in Amstelveen, Netherlands, in 1998. The new variety is a mutation of ‘Britt’ (unpatented). The new variety differs from its parent through its yellow/pale yellow flower color and a similar variety, ‘Rhianne’ (unpatented) in its flower color and peduncle color as follows:

| | Rhianne | Nadine |
|---|---|---|
| Middle of lower side, | Orange Group 25D | Yellow-Orange Group 18B |
| outer tepal | | |
| Middle of lower side, | Orange Group 24D | Yellow-Orange Group 21D |
| inner tepal | | |
| Peduncle | Reddish | Greenish |

The new variety was first asexually reproduced by cuttings in Aalsmeer in February of 1998. The new variety has been trial and field tested and has been found to retain its distinctive characteristics and remain true to type through successive propagations.

The new variety, when grown in a glass greenhouse has a response time for rooting from a terminal cutting of 14 days.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions by cuttings in a controlled greenhouse environment. Color references are primarily to the R.H.S. Colour Chart of The Royal Horticultural Society of London.

### PLANT

Classification:

Botanical.—Begonia Elatior.

Commercial.—Begonia.

Form: Upright.

Size: About 40 cm tall from top of media surface; 30 cm wide in 15 cm container 10 weeks after planting, with pinching.

Growth: Moderate.

Time to initiate roots: About 14 days at 22° C.

Time to develop roots: About 18 days at 22° C.

Rooting habit: Fine, fibrous, and well-branched.

Lateral Branches:

Appearance/aspect.—Cylindrical; upright to somewhat outwardly angled.

Strength.—Very good.

Length.—About 19 cm.

Diameter.—About 1 cm.

Internode length.—About 3.8 cm.

Texture.—Mostly smooth with sparse small white hairs; fleshy.

Leaves:

Arrangement.—Alternate.

Shape.—Rounded with acute apex and cordate base with overlap.

Size.—14 cm wide; 15 cm long.

Quantity.—About 7 per lateral branch.

Stipules.—2 per leaf sized 5 mm×5 mm.

Margin.—Bi-crenate.

Texture.—Smooth; shiny and leathery.

Color.—Upper surface: Green Group 136A. Lower surface: Green Group 137C.

Venation.—Palmate; light green.

Petiole.—About 4.5 cm long; 5 mm in diameter.

### FLOWERS

Habit: Continuous flowering.

Size: 3.5 mm in diameter; 1 cm deep.

Tepals:

Arrangement: Double.—14-20 tepals per flower.

Size.—2.5 cm long; 3.5 cm wide.

Margin.—Entire.

Flower buds: About 1.8 cm long (in autumn); about 1.3 cm wide.

Peduncles:

Angle.—Mostly erect.

Strength.—Strong but flexible.

Length.—About 3.5-4 cm.

Color.—Yellow-Green Group 146B.

Pedicels:

Angle.—About 30 to 45° from vertical.

Strength.—Strong but flexible.

Length.—About 1 cm.

Color.—Yellow-Green Group 146B.

Bracts:

Arrangement.—Two; opposite.

Shape.—Very broadly cordate; acute apex.

Margin.—Entire.

Color.—Light green.

Color:

Upper surface.—Outer petals: Margin upper side: Yellow-Orange Group 16C. Middle upper side: Yellow-Orange Group 19C. Inner petals: Margin upper side: Yellow-Orange Group 16A. Middle upper side: Yellow-Orange Group 21B.

Lower surface.—Yellow-Orange Group 18B deepening to Yellow-Orange Group 21D.

### REPRODUCTIVE ORGANS

Stamens: None observed.

Pistils: None observed.

Postproduction longevity:

Flowers.—3-4 weeks.

Plants.—7-8 weeks.

Disease resistance: Mildew tolerant.

Seed Production: Seed production has not been observed as reproductive organs are not forned.

I claim:

1. A new and distinct variety of begonia plant substantially as shown and described.
